(19) United States
(12) Patent Application Publication
(10) Pub. No.: US 2026/0240889 A1
(43) Pub. Date: Aug. 20, 2026

(54) COMPOSITIONS AND METHODS FOR PREVENTION AND TREATMENT OF GENETIC DISEASE

(71) Applicant: The Children's Medical Center Corporation, Boston, MA (US)

(72) Inventor: Dario O. Fauza, Boston, MA (US)

(73) Assignee: The Children's Medical Center Corporation, Boston, MA (US)

(21) Appl. No.: 19/648,413

(22) Filed: Apr. 15, 2026

Related U.S. Application Data
(63) Continuation of application No. PCT/US2024/051733, filed on Oct. 17, 2024.
(60) Provisional application No. 63/545,030, filed on Oct. 20, 2023.

(51) Int. Cl. A61K 31/7105; A61K 9/00; A61K 9/1272; A61K 9/51; A61K 38/57; A61P 11/00; C12N 15/88

(52) U.S. Cl. CPC A61K 31/7105; A61K 9/0034; A61K 9/1272; A61K 9/5123; A61K 38/57; A61P 11/00; C12N 15/88

(57) ABSTRACT

Methods and compositions for the transamniotic delivery of mRNA encoding alpha-1 antitrypsin protein to a fetus for the prenatal treatment of alpha-1 antitrypsin deficiency.

### CROSS-REFERENCE TO RELATED APPLICATION

The present application is a continuation under 35 U.S.C. § 111 (a) of PCT International Patent Application No. PCT/US2024/051733, filed Oct. 17, 2024, designating the United States and published in English, which claims priority to and the benefit of U.S. Provisional Application No. 63/545,030, filed Oct. 20, 2023, which is hereby incorporated by reference in its entirety.

### SEQUENCE LISTING

The instant application contains a Sequence Listing which has been submitted electronically as an XML file and is hereby incorporated by reference in its entirety. The Sequence Listing, created on Oct. 22, 2024, is named “167705-034501PCT_SL.xml” and is 6,473 bytes in size.

### BACKGROUND OF THE INVENTION

Alpha-1 antitrypsin (AAT) is produced primarily in the liver and, to a lesser extent, also in the lungs. This protein has numerous anti-inflammatory and immunomodulatory effects both associated with and independent of its modulation of various proteases. First described in 1963 by Laurell and Eriksson, AAT deficiency (AATD) has since been identified as an autosomal codominant disease resulting from mutations in the SerpinA1 gene sequence. It is one of the most common inherited metabolic disorders affecting 1:2,000-5,000 people at any age, including the perinatal period. Liver and obstructive lung disease are among its chief manifestations, for which current therapies are cumbersome and of limited benefit.

Alpha-1 antitrypsin deficiency (AATD) manifests at any age, including the perinatal period. Researchers sought to determine whether the transamniotic route could be a viable alternative for administering AAT mRNA to the fetus.

### SUMMARY OF THE INVENTION

The invention features compositions and methods that are useful for transamniotic delivery of mRNA encoding alpha-1 antitrypsin (AAT) to a fetus for the prenatal treatment of alpha-1 antytrpsin deficiency (AATD).

In one aspect, the invention features a method for treating alpha-1 antitrypsin deficiency (AATD) in a developing fetus, the method involving: administering to the amniotic fluid surrounding a developing fetus an mRNA molecule encoding an alpha-1 antitrypsin (AAT) polypeptide, thereby treating AATD in the developing fetus.

In another aspect, the invention features a method for treating a disease or disorder associated with an AAT deficiency in a developing fetus, the method involving: administering to the amniotic fluid surrounding a developing fetus an mRNA molecule encoding an AAT polypeptide, thereby treating the disease or disorder associated with AAT deficiency in the developing fetus.

In another aspect, the invention features a kit suitable for use in carrying out the method of any previous aspect, wherein the kit includes an mRNA described and/or claimed herein.

In another aspect, the invention features a method for delivering one or more mRNA molecules to the bloodstream of a developing fetus, the method involving: administering to the amniotic fluid surrounding the developing fetus an mRNA molecule encoding a polypeptide that has reduced expression and/or activity in the developing fetus relative to a healthy developing fetus.

In various embodiments of any of the above aspects or any other aspect of the invention described herein, the mRNA is translated in the lungs and/or liver of the fetus to produce the AATD polypeptide in lung cells of the fetus. In various embodiments of any of the above aspects, the method involves reducing the incidence of chronic obstructive pulmonary disease in the fetus or the child developed from the fetus. In various embodiments of any of the above aspects, the disease or disorder is prematurity or congenital AAT deficiency. In various embodiments of any of the above aspects, the mRNA is translated in the liver of the fetus to produce the AAT polypeptide in liver cells of the fetus. In various embodiments of any of the above aspects, the mRNA is translated in lung cells of the fetus to produce the AAT polypeptide in airway and/or pulmonary cells of the fetus. In various embodiments of any of the above aspects, the method is associated with an increase in levels of AAT in the amniotic fluid. In various embodiments of any of the above aspects, the mRNA is administered using a lipid nanoparticle or a lipopolyplex. In various embodiments of any of the above aspects, the lipopolyplex includes a cationic polymer. In various embodiments of any of the above aspects, the method is associated with increased life expectancy for the child developed from the fetus. In various embodiments of any of the above aspects, the mRNA is codon-optimized. In various embodiments of any of the above aspects, the polypeptide is a human polypeptide. In various embodiments of any of the above aspects, the fetus is a mammalian fetus. In various embodiments of any of the above aspects, the fetus is a human fetus. In various embodiments of any of the above aspects, the mRNA is administered prior to 10 weeks of pregnancy. In various embodiments of any of the above aspects, the mRNA is administered prior to 5 weeks of pregnancy. In various embodiments of any of the above aspects, the mRNA is administered after 10 weeks of pregnancy. In various embodiments of any of the above aspects, the administering is associated with the presence of and/or an increase in levels of the mRNA in a tissue of the fetus. In various embodiments of any of the above aspects, the tissue includes liver, stomach, intestines, pancreas, spleen, thymus, lymph nodes, brain, meninges, heart, blood vessels, lungs, airways, kidneys, ureters, urethra, ovaries, testicles, genitalia, skin, skin annexes, muscle, bone, cartilage, bone marrow, eyes, ears, mouth, nose, pharynx, larynx, vitreous body, cerebrospinal fluid, and peripheral blood. In various embodiments of any of the above aspects, the tissue includes liver, stomach, intestines, lungs, and/or blood. In various embodiments of any of the above aspects, the administration is associated with the presence of and/or an increase in levels of the mRNA in amniotic fluid, amnion, chorion, umbilical cord, and/or placenta associated with the fetus.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof. In some embodiments, the agent is an mRNA molecule. In some instances, the mRNA molecule encodes alpha-1 antitrypsin.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “alpha-1 antytrypsin (AAT) polypeptide” is meant a polypeptide or fragment thereof having at least 85% amino acid sequence identity to NCBI reference sequence NO.: NP_000286.3 or a fragment thereof capable of mediating transport of ions across a membrane. An exemplary AAT polypeptide sequence is provided below:

| >NP_000286.3 | |
|---|---|
| (SEQ ID NO: 1) | |
| 1 | mpssvswgil llaglcclvp vslaedpqgd aaqktdtshh dqdhptfnki tpnlaefafs |
| 61 | lyrqlahqsn stniffspvs iatafamlsl gtkadthdei leglnfnlte ipeaqihegf |
| 121 | qellrtlnqp dsqlqlttgn glflseglkl vdkfledvkk lyhseaftvn fgdteeakkq |
| 181 | indyvekgtq gkivdlvkel drdtvfalvn yiffkgkwer pfevkdteee dfhvdqvttv |
| 241 | kvpmmkrlgm fniqhckkls swvllmkylg nataifflpd egklqhlene lthdiitkfl |
| 301 | enedrrsasl hlpklsitgt ydlksvlgql gitkvfsnga dlsgvteeap lklskavhka |
| 361 | vltidekgte aagamfleai pmsippevkf nkpfvflmie qntksplfmg kvvnptqk |

By “AAT polynucleotide” is meant a nucleic acid molecule encoding a AAT polypeptide, or a fragment thereof. An exemplary AAT polynucleotide sequence is provided at NCBI reference sequence NO.: NM_001127707.2, which is reproduced below:

| (SEQ ID NO: 2) | |
|---|---|
| 1 | agagtcctga gctgaaccaa gaaggaggag ggggtcgggc ctccgaggaa ggcctagccg |
| 61 | ctgctgctgc caggaattcc aggttggagg ggcggcaacc tcctgccagc cttcaggcca |
| 121 | ctctcctgtg cctgccagaa gagacagagc ttgaggagag cttgaggaga gcaggaaagc |
| 181 | ctcccccgtt gcccctctgg atccactgct taaatacgga cgaggacagg gccctgtctc |
| 241 | ctcagcttca ggcaccacca ctgacctggg acagtgaatc gacaatgccg tcttctgtct |
| 301 | cgtggggcat cctcctgctg gcaggcctgt gctgcctggt ccctgtctcc ctggctgagg |
| 361 | atccccaggg agatgctgcc cagaagacag atacatccca ccatgatcag gatcacccaa |
| 421 | ccttcaacaa gatcaccccc aacctggctg agttcgcctt cagcctatac cgccagctgg |
| 481 | cacaccagtc caacagcacc aatatcttct tctccccagt gagcatcgct acagcctttg |
| 541 | caatgctctc cctggggacc aaggctgaca ctcacgatga aatcctggag ggcctgaatt |
| 601 | tcaacctcac ggagattccg gaggctcaga tccatgaagg cttccaggaa ctcctccgta |
| 661 | ccctcaacca gccagacagc cagctccagc tgaccaccgg caatggcctg ttcctcagcg |
| 721 | agggcctgaa gctagtggat aagtttttgg aggatgttaa aaagttgtac cactcagaag |
| 781 | ccttcactgt caacttcggg gacaccgaag aggccaagaa acagatcaac gattacgtgg |
| 841 | agaagggtac tcaagggaaa attgtggatt tggtcaagga gcttgacaga gacacagttt |
| 901 | ttgctctggt gaattacatc ttctttaaag gcaaatggga gagacccttt gaagtcaagg |
| 961 | acaccgagga agaggacttc cacgtggacc aggtgaccac cgtgaaggtg cctatgatga |
| 1021 | agcgtttagg catgtttaac atccagcact gtaagaagct gtccagctgg gtgctgctga |
| 1081 | tgaaatacct gggcaatgcc accgccatct tcttcctgcc tgatgagggg aaactacagc |
| 1141 | acctggaaaa tgaactcacc cacgatatca tcaccaagtt cctggaaaat gaagacagaa |
| 1201 | ggtctgccag cttacattta cccaaactgt ccattactgg aacctatgat ctgaagagcg |
| 1261 | tcctgggtca actgggcatc actaaggtct tcagcaatgg ggctgacctc tccggggtca |
| 1321 | cagaggaggc acccctgaag ctctccaagg ccgtgcataa ggctgtgctg accatcgacg |
| 1381 | agaaagggac tgaagctgct ggggccatgt ttttagaggc catacccatg tctatccccc |
| 1441 | ccgaggtcaa gttcaacaaa ccctttgtct tcttaatgat tgaacaaaat accaagtctc |
| 1501 | ccctcttcat gggaaaagtg gtgaatccca cccaaaaata actgcctctc gctcctcaac |
| 1561 | ccctcccctc catccctggc cccctccctg gatgacatta aagaagggtt gagctggtcc |
| 1621 | ctgcctgcat gtgactgtaa atccctccca tgttttctct gagtctccct ttgcctgctg |
| 1681 | aggctgtatg tgggctccag gtaacagtgc tgtcttcggg ccccctgaac tgtgttcatg |
| 1741 | gagcatctgg ctgggtaggc acatgctggg cttgaatcca ggggggactg aatcctcagc |
| 1801 | ttacggacct gggcccatct gtttctggag ggctccagtc ttccttgtcc tgtcttggag |
| 1861 | tccccaagaa ggaatcacag gggaggaacc agataccagc catgacccca ggctccacca |
| 1921 | agcatcttca tgtccccctg ctcatccccc actccccccc acccagagtt gctcatcctg |
| 1981 | ccagggctgg ctgtgcccac cccaaggctg ccctcctggg ggccccagaa ctgcctgatc |
| 2041 | gtgccgtggc ccagttttgt ggcatctgca gcaacacaag agagaggaca atgtcctcct |
| 2101 | cttgacccgc tgtcacctaa ccagactcgg gccctgcacc tctcaggcac ttctggaaaa |
| 2161 | tgactgaggc agattcttcc tgaagcccat tctccatggg gcaacaagga cacctattct |
| 2221 | gtccttgtcc ttccatcgct gccccagaaa gcctcacata tctccgttta gaatcaggtc |
| 2281 | ccttctcccc agatgaagag gagggtctct gctttgtttt ctctatctcc tcctcagact |
| 2341 | tgaccaggcc cagcaggccc cagaagacca ttaccctata tcccttctcc tccctagtca |
| 2401 | catggccata ggcctgctga tggctcagga aggccattgc aaggactcct cagctatggg |
| 2461 | agaggaagca catcacccat tgacccccgc aacccctccc tttcctcctc tgagtcccga |
| 2521 | ctggggccac atgcagcctg acttctttgt gcctgttgct gtccctgcag tcttcagagg |
| 2581 | gccaccgcag ctccagtgcc acggcaggag gctgttcctg aatagcccct gtggtaaggg |
| 2641 | ccaggagagt ccttccatcc tccaaggccc tgctaaagga cacagcagcc aggaagtccc |
| 2701 | ctgggcccct agctgaagga cagcctgctc cctccgtctc taccaggaat ggccttgtcc |
| 2761 | tatggaaggc actgccccat cccaaactaa tctaggaatc actgtctaac cactcactgt |
| 2821 | catgaatgtg tacttaaagg atgaggttga gtcataccaa atagtgattt cgatagttca |
| 2881 | aaatggtgaa attagcaatt ctacatgatt cagtctaatc aatggatacc gactgtttcc |
| 2941 | cacacaagtc tcctgttctc ttaagcttac tcactgacag cctttcactc tccacaaata |
| 3001 | cattaaagat atggccatca ccaagccccc taggatgaca ccagacctga gagtctgaag |
| 3061 | acctggatcc aagttctgac ttttccccct gacagctgtg tgaccttcgt gaagtcgcca |
| 3121 | aacctctctg agccccagtc attgctagta agacctgcct ttgagttggt atgatgttca |
| 3181 | agttagataa caaaatgttt atacccatta gaacagagaa taaatagaac tacatttctt |
| 3241 | gca |

In embodiments, the AAT polynucleotide is a DNA molecule, mRNA molecule, or hybrid DNA/RNA molecule.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Non-limiting examples of diseases that may be treated using the compositions and/or methods provided herein include chronic obstructive pulmonary disease, cirrhosis, chronic liver disease, hepatocellular carcinoma, emphysema, and an alpha-1 antitrypsin (AAT) deficiency.

By “effective amount” is meant the amount of an agent required to ameliorate the symptoms of a disease relative to an untreated patient. In some embodiments, the agent is an mRNA encoding AAT. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state.

“Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “lipopolyplex” is meant a structure for encapsulation and delivery of an agent into an organism and comprises a nucleic acid molecule, cationic lipids, and cationic polymers. Non-limiting examples of lipopolyplex compositions include those described in To et al., Expert Opinion on Drug Discovery 16, 1307-1313, 2021, the disclosure of which is incorporated herein in its entirety for all purposes.

By “lipid nanoparticle” is meant a small particle comprising lipids that is suitable for delivering an agent to a cell. In embodiments, the cell is in a subject. In embodiments, the lipid nanoparticle comprises (e.g., encapsulates) the agent. In various embodiments, the agent is an mRNA molecule. As used herein, the term lipid encompasses triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e.g. cetyl palmitate).

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule (e.g., mRNA or an RNA molecule) that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. In some instances, the nucleic acid molecule is an mRNA molecule. In embodiments, the mRNA molecule comprises a 5′ cap, a poly(A) tail, a 3′ untranslated region, and/or a 5′ untranslated region. Components of mRNA molecules suitable for use in the methods of the present invention include those described in Sahin, et al., “mRNA-based therapeutics-developing a new class of drugs,” Nature Reviews Drug Discovery, 13:759-780 (2014), the disclosure of which is incorporated herein by reference in its entirety for all purposes.

In embodiments, the polynucleotides provided herein contain one or more modifications or analogs. For example, in some embodiments a polynucleotide contains one or more analogs (e.g., altered backbone, sugar, or nucleobase). Some non-limiting examples of analogs include 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine.

In embodiments, the polynucleotide contains a modified backbone and/or linkages (e.g., between adjacent nucleosides). Non-limiting examples of modified backbones include those that contain a phosphorus atom in the backbone and those that do not contain a phosphorus atom in the backbone. Non-limiting examples of modified backbones include phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

In embodiments, a polynucleotide contains short chain alkyl or cycloalkyl linkages (e.g., between adjacent nucleosides), mixed heteroatom and alkyl or cycloalkyl internucleoside linkages, or one or more short chain heteroatomic or heterocyclic internucleoside linkages. In embodiments, a polynucleotide includes one or more of the following: morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.
In embodiments, a polynucleotide contains a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. In embodiments, the backbone in PNA compounds contains two or more linked aminoethylglycine units that give PNA an amide containing backbone. Heterocyclic base moieties can be bound directly or indirectly to aza nitrogen atoms of the amide portion of the backbone.
In embodiments, a polynucleotide contains a morpholino backbone structure. For example, a nucleic acid can contain a 6-membered morpholino ring in place of a ribose ring. In some of these embodiments, a phosphorodiamidate or other non-phosphodiester internucleoside linkage can replace a phosphodiester linkage.

A polynucleotide can contain linked morpholino units having heterocyclic bases attached to the morpholino ring. Linking groups can link morpholino monomeric units. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). In some instances, the furanose ring normally present in a nucleic acid molecule is replaced with a cyclohexenyl ring. CeNA DMT protected phosphoramidite monomers can be prepared and used for oligomeric compound synthesis using phosphoramidite chemistry. In some cases, incorporation of CeNA monomers into a nucleic acid chain increases the stability of a DNA/RNA hybrid. CeNA oligoadenylates can form complexes with nucleic acid complements with similar stability to the native complexes. In embodiments, a polynucleotide contains Locked Nucleic Acids (LNAs) in which the 2′-hydroxyl group is linked to the 4′ carbon atom of the sugar ring thereby forming a 2′-C, 4′-C-oxymethylene linkage, thereby forming a bicyclic sugar moiety. The linkage can be a methylene (—CH₂), group bridging the 2′ oxygen atom and the 4′ carbon atom wherein n is 1 or 2. LNA and LNA analogs can display very high duplex thermal stabilities with complementary nucleic acid (Tₘ=+3 to +10° C.), stability towards 3′-exonucleolytic degradation and good solubility properties.

In embodiments, a polynucleotide contains nucleobase modifications (often referred to simply as “base modifications”) or substitutions. In embodiments, unmodified nucleobases include one or more of the purine bases, (e.g., adenine (A) and guanine (G)), and/or the pyrimidine bases, (e.g., thymine (T), cytosine (C) and uracil (U)). Non-limiting examples of modified nucleobases include nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further non-limiting examples of modified nucleobases include tricyclic pyrimidines such as phenoxazine cytidine (1H-pyrimido (5,4-b) (1,4) benzoxazin-2 (3H)-one), phenothiazine cytidine (1H-pyrimido (5,4-b) (1,4)benzothiazin-2 (3H)-one), G-clamps such as a substituted phenoxazine cytidine (e.g., 9-(2-aminoethoxy)-H-pyrimido (5,4-(b) (1,4)benzoxazin-2 (3H)-one), phenothiazine cytidine (1H-pyrimido (5,4-b) (1,4)benzothiazin-2 (3H)-one), G-clamps such as a substituted phenoxazine cytidine (e.g., 9-(2-aminoethoxy)-H-pyrimido (5,4-(b) (1,4)benzoxazin-2 (3H)-one), carbazole cytidine (2H-pyrimido (4,-b) indol-2-one), pyridoindole cytidine (H-pyrido (3′,2′: 4,5) pyrrolo[2,3-d]pyrimidin-2-one).

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 g/ml denatured salmon sperm DNA (ssDNA). In a most preferred embodiment, hybridization will occur at 42° C. in 250 mM NaCl, 25 mM trisodium citrate, 1% SDS, 50% formamide, and 200 g/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York. By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides an illustration showing an overview of the experimental protocol used in the below examples. hAAT=human Alpha-1 Antitrypsin; E=gestational day (term=E21).

FIGS. 2A-2B provide plots showing alpha-1 antitrypsin (AAT) protein levels in fetal rats at different gestational points over a 21-day term, where E=gestational day. FIG. 2A provides a plot showing human AAT protein levels in the lungs of fetal rats from both the human AAT mRNA group and the control group. FIG. 2B provides a plot showing human AAT protein levels in the livers of fetal rats from both the human AAT mRNA group and the control group.

### DETAILED DESCRIPTION OF THE INVENTION

The invention features compositions and methods that are useful for transamniotic delivery of mRNA encoding alpha-1 antitrypsin (AAT) to a fetus for the prenatal treatment of alpha-1 antytrpsin deficiency (AATD).

The invention of the disclosure is based, at least in part, upon the discovery that packaged exogenous mRNA can be incorporated by the fetus after simple intra-amniotic administration in a rodent model. As described further in the Examples provided herein, experiments were undertaken to examine the pharmacokinetics of exogenous encapsulated mRNA delivered transamniotically, utilizing a healthy rat model. The pattern and chronology of mRNA incorporation were compatible with transplacental hematogenous routing, as well as with fetal swallowing/aspiration. The invention of the disclosure is also based, at least in part, upon the discovery that encapsulated exogenous mRNA encoding for AAT was incorporated and translated by fetal lung cells after simple intra-amniotic injection in a healthy rat model.

Many prenatally diagnosable diseases involve abnormal or missing proteins and thus could be conceivably amenable to mRNA-based therapies, along with certain gestational or perinatal infections for which mRNA-based vaccines could also be beneficial (Alapati et al., Science Translational Medicine 11, 2019; Antony et al., Molecular and Cellular Pediatrics 2, 11, 2015; Derosa et al., Gene Therapy 23, 699-707, 2016; Fazio et al., Gene therapy 11, 544-551, Kang et al., Nature Communications 11, 3929, 2020; Massaro et al., Nature Medicine 24, 1317-1323, 2018; Riley et al., Science Advances 7, 2021; Rizzi et al, Vaccine 23, 4273-4282, 2005, the disclosures of which are incorporated herein by reference in their entireties for all purposes).

### Transamniotic Nucleic Acid Therapy

The invention provides transamniotic nucleic acid therapy (TRANAT) methods for treating and/or preventing an alpha-1 antitrypsin deficiency (AATD) or symptoms thereof. Thus, one embodiment is a method of treating a fetus suffering from or susceptible to a disease (e.g., AATD), infection, and/or disorder or symptom thereof. The methods involve administering to the amniotic fluid a therapeutically effective amount of an mRNA. The mRNA is administered by intra-amniotic injection. The method involves the step of administering to the fetus a therapeutic amount of an amount of an mRNA sufficient to treat the disease or disorder or symptom thereof, under conditions such that the disease, infection, and/or disorder is treated and/or prevented. The mRNA can be delivered using common microencapsulation methods utilizing lipid nanoparticle delivery such as, for example, those described in: “Hou et al., “Lipid Nanoparticles for MRNA Delivery,” Nature Reviews Materials, December 2021, Vol. 6, No. 12, pp. 1078-1094.” In some embodiments, the method of delivery can include the use of lipopolyplexes such as, for example, those described in: “Bofinger et al., “Development of Lipopolyplexes for Gene Delivery: A Comparison of the Effects of Differing Modes of Targeting Peptide Display on the Structure and Transfection Activities of Lipopolyplexes,” Journal of Peptide Science, 2018, Vol. 24, No. 12, p. e3131.”

The methods herein include administering to the amniotic fluid surrounding a fetus an effective amount of an mRNA, or a composition described herein to produce such effect. Identifying a subject in need of such treatment can be in the judgment of a subject or a health care professional and can be subjective (e.g., opinion) or objective (e.g., measurable by a test or diagnostic method).

The therapeutic methods of the invention (which include prophylactic treatment) in general comprise administration of a therapeutically effective amount of an mRNA to the amniotic fluid surrounding a fetus. In embodiments, the fetus is a human fetus. Such treatment will be suitably administered to fetuses, suffering from, having, susceptible to, or at risk for a disease, infection, and/or disorder, or symptom thereof, before and/or after birth. Determination of those subjects “at risk” can be made by any objective or subjective determination by a diagnostic test or opinion of a subject or health care provider (e.g., genetic test, enzyme or protein marker, Marker (as defined herein), family history, and the like).

In some embodiments, an mRNA is administered to the amniotic fluid in an amount of about or at least about 0.01 μg, 0.1 μg, 0.5 μg, 1 μg, 5 μg, 10 μg, 15 μg, 20 μg, 25 μg, 100 μg, 250 μg, 500 μg, 750 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 100 mg, 200 mg, 300 mg, 400 mg, 500 mg, 600 mg, 700 mg, 800 mg, 900 mg, or 1,000 mg. In some embodiments, an mRNA is administered to the amniotic fluid in an amount of no more than about 0.01 μg, 0.1 μg, 0.5 μg, 1 μg, 5 μg, 10 μg, 15 μg, 20 μg, 25 μg, 100 μg, 250 μg, 500 μg, 750 μg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 200 mg, 300 mg, 400 mg, 500 mg, 600 mg, 700 mg, 800 mg, 900 mg, or 1,000 mg, 5

Generally, doses of mRNAs of the invention will be from about 0.0001 mg/kg fetal weight per day to about 1000 mg/kg fetal weight per administration. It is expected that doses ranging from about 1 to about 50 mg/kg will be suitable. In an embodiment, an mRNA is administered to the amniotic fluid in amounts sufficient to deliver about or at least about 0.1 mg/kg fetal weight, 0.0001 mg/kg fetal weight, 0.001 mg/kg fetal weight, 0.01 mg/kg fetal weight, 0.1 mg/kg fetal weight, 0.5 mg/kg fetal weight, 1 mg/kg fetal weight, 2 mg/kg fetal weight, 3 mg/kg fetal weight, 4 mg/kg fetal weight, 5 mg/kg fetal weight, 6 mg/kg fetal weight 7 mg/kg fetal weight, 8 mg/kg fetal weight, 9 mg/kg fetal weight, or 10 mg/kg fetal weight of the polynucleotide to the fetus. In an embodiment, an mRNA is administered to the amniotic fluid in amounts sufficient to deliver no more than about 0.0001 mg/kg fetal weight, 0.001 mg/kg fetal weight, 0.01 mg/kg fetal weight, 0.1 mg/kg fetal weight, 05 mg/kg fetal weight, 1 mg/kg fetal weight, 2 mg/kg fetal weight, 3 mg/kg fetal weight, 4 mg/kg fetal weight, 5 mg/kg fetal weight, 6 mg/kg fetal weight 7 mg/kg fetal weight, 8 mg/kg fetal weight, 9 mg/kg fetal weight, or 10 mg/kg fetal weight of the polynucleotide to the fetus. In the event that a response in a subject is insufficient at the initial doses applied, higher doses (or effectively higher doses) may be employed to the extent that subject tolerance permits. Multiple doses per day are contemplated to achieve appropriate systemic levels of an agent and/or compositions of the invention.

In some embodiments, administration of an mRNA of the invention to the amniotic fluid is associated with a reduction in the intensity, severity, or frequency, or delays the onset of a disorder, disease, and/or deficiency (e.g., an alpha-1 antitrypsin deficiency) in the fetus before and/or after birth (i.e., prenatal or perinatal).

It can be advantageous to administer the mRNA at a particular time during the development of the fetus. In embodiments, the mRNA is administered prior to, at, after, and/or until about 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 25 weeks, 30 weeks, 35 weeks, or 40 weeks of pregnancy. In embodiments, it is advantageous to administer the mRNA prior to 20 weeks of pregnancy.

In embodiments, the trans-amniotic delivery of the mRNA to the amniotic fluid results in the presence of and/or an increase in levels of the mRNA in the fetal blood plasma. In embodiments, the trans-amniotic delivery of the mRNA to the amniotic fluid results in an increase in levels of the mRNA in fetal tissues (e.g., thymus, spleen, brain, and/or bone marrow, and/or others, given the presence of the mRNA in the fetal blood plasma). Further non-limiting examples of tissues include liver, stomach, intestines, pancreas, spleen, thymus, lymph nodes, brain, meninges, heart, blood vessels, lungs, airways, kidneys, ureters, urethra, ovaries, testicles, genitalia, skin, skin annexes, muscle, bone, cartilage, bone marrow, eyes, ears, mouth, nose, pharynx, larynx, vitreous body, cerebrospinal fluid, and peripheral blood.

A skilled practitioner will readily be able to identify suitable methods for detecting and measuring levels of an mRNA in the amniotic fluid or in any samples taken from a fetus or from a neonate that received such treatment before birth. For example, quantitative real-time polymerase chain reaction (qRT-PCR) is one example of many techniques available for measuring mRNA levels in biological samples such as, to provide non-limiting examples, serum, bone marrow, spleen tissue, thymus tissue, and brain tissue. The biological samples can be collected from the fetus and/or the mother.

mRNA

The methods of the invention involve administering an mRNA to the amniotic fluid surrounding a fetus. In embodiments, the mRNA is encapsulated in a lipid nanoparticle or lipopolyplex to enhance stability, tolerability, or solubility; or decrease the likelihood of eliciting an immune response in the target. Additionally, in some embodiments, the invention may include modified nucleobases for masking the nucleic acid payload from the immune system and decreasing the chances of an unintended immune response or triggering anaphylaxis. In some embodiments, such modifications can include, but are not limited to those modifications described herein, such as pseudouridine, thiouridine, and 5-methylcytidine. In some embodiments, the mRNA is single-stranded. In further embodiments, the mRNA can include untranslated regions (UTRs) at the 5′ and 3′ end to enhance stability. In some embodiments, the mRNA includes one or more of a 3′ poly(A) tail, a 5′ cap, UTRs, and a protein-encoding region. In embodiments, the mRNA molecule comprises a 5′ cap, a poly(A) tail, a 3′ untranslated region, and/or a 5′ untranslated region. Components of mRNA molecules suitable for use in the methods of the present invention include those described in Sahin, et al., “mRNA-based therapeutics-developing a new class of drugs,” Nature Reviews Drug Discovery, 13:759-780 (2014), the disclosure of which is incorporated herein by reference in its entirety for all purposes.

In some embodiments, a DNA template can be used to produce synthetic mRNA. In further embodiments, in vitro transcription technology is used to transcribe the sequence of interest into mRNA, which can be purified for delivery into a target or fetus. In some embodiments, mRNA synthesis takes place in a cell-free system. In other embodiments, cells engineered to express an mRNA sequence of interest can be cultivated and purified. In some embodiments, the engineered cells have been transfected with a plasmid containing a DNA template for the mRNA sequence of interest.

In some embodiments, the mRNA being administered to a subject has been modified to improve translation efficiency in a target cell or has been modified to augment expression, which can include optimization of stop and start codons for efficient elongation and termination of translation. This optimization can include the replacement of similar codons with a major codon that is preferred by highly expressed genes or a codon that is decoded by more tRNA than similar codons. Methods for codon optimization are well known and include those methods described, for example, in Mauro and Chappel, “A critical analysis of codon optimization in human therapeutics,” Trends Mol Med., 20:604-613 (2014), the disclosure of which is incorporated herein by reference in its entirety for all purposes.

### Pharmaceutical Compositions

The invention provides compositions comprising a polynucleotide (e.g., mRNA) for use in methods for preventing and/or treating alpha-1 antitrypsin deficiencies in a developing fetus. The compositions should be sterile and contain a therapeutically effective amount of the polynucleotide in a unit of weight or volume suitable for administration to a subject.

Agents (e.g., mRNA) of the invention may be administered within a pharmaceutically-acceptable diluent, carrier, or excipient, in unit dosage form. In some embodiments, the agents of the invention may be administered through injection of lipid nanoparticles or lipoplexes containing mRNA of interest.

Suitable pharmaceutically acceptable carriers include but are not limited to water, salt solutions (e.g., NaCl), saline, buffered saline, alcohols, glycerol, ethanol, gum arabic, benzyl alcohols, polyethylene glycols, gelatin, carbohydrates such as lactose, amylose or starch, sugars such as mannitol, sucrose, or others, dextrose, magnesium stearate, viscous paraffin, fatty acid esters, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, and/or coloring substances and the like) which do not deleteriously react with the active compounds or interfere with their activity. In an embodiment, a water-soluble carrier suitable for intravenous administration is used.

A suitable pharmaceutical composition, if desired, can also contain minor amounts of wetting or emulsifying agents, or pH buffering agents. A composition can be a liquid solution.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful delivery systems for agents of the invention include ethylene-vinyl acetate copolymer particles, osmotic pumps, implantable infusion systems, and liposomes.

The formulations can be administered to human patients in therapeutically effective amounts. The preferred dosage of a polynucleotide the invention is likely to depend on such variables as the volume of the amniotic cavity, or the nature of a disorder being treated. In an embodiment, the polynucleotide is administered more than once in a given pregnancy.

A pharmaceutical composition or medicament can be formulated in accordance with the routine procedures as a pharmaceutical composition adapted for administration to human beings. For example, in some embodiments, a composition for intravenous administration typically is a solution in sterile isotonic aqueous buffer. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline, or dextrose/water. Where the composition is administered by injection, an ampule of sterile water for injection or saline can be provided so that the ingredients may be mixed prior to administration.

### Kits

The invention provides kits for use in preventing and/or treating AATD in a developing fetus. In one embodiment, the kit contains a pharmaceutical composition containing an mRNA suitable for us in preventing and/or treating AATD. In an embodiment, the kit contains equipment (e.g., hypodermic needles and syringes) to aid in administration of compositions of the invention to amniotic fluid surrounding a fetus.

Optionally, the kit includes directions for administering the pharmaceutical composition to amniotic fluid. In other embodiments, the kit comprises a sterile container which contains the pharmaceutical composition. Such containers can be boxes, ampules, bottles, vials, tubes, bags, pouches, blister-packs, or other suitable container form known in the art. Such containers can be made of plastic, glass, laminated paper, metal foil, or other materials suitable for holding compositions containing mRNA. The instructions will generally include information about the pharmaceutical composition (e.g., safety information, recommended doses, and the like) and how to administer the composition to the amniotic fluid surrounding a fetus. In other embodiments, the instructions include at least one of the following: description of the mRNA; methods for using the enclosed materials; precautions; warnings; indications; clinical or research studies; and/or references. The instructions may be printed directly on the container (when present), or as a label applied to the container, or as a separate sheet, pamphlet, card, or folder supplied in or with the container.

The practice of the invention employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for specific embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Fetal hAAT Production

The encapsulation efficiency of the hAAT into the lipopolyplex was 96%. Overall fetal survival was 79% (110/139), but significantly higher in the mRNA group than in the control group (respectively 85% vs. 69%, p=0.012). There was no maternal mortality. The following number of fetuses were examined at each time point for a total n=110: at E18-mRNA n=21, control n=9; at E19-mRNA n=23, control n=6; at E20-mRNA n=21, control n=5; at E21-mRNA n=19, control n=6.

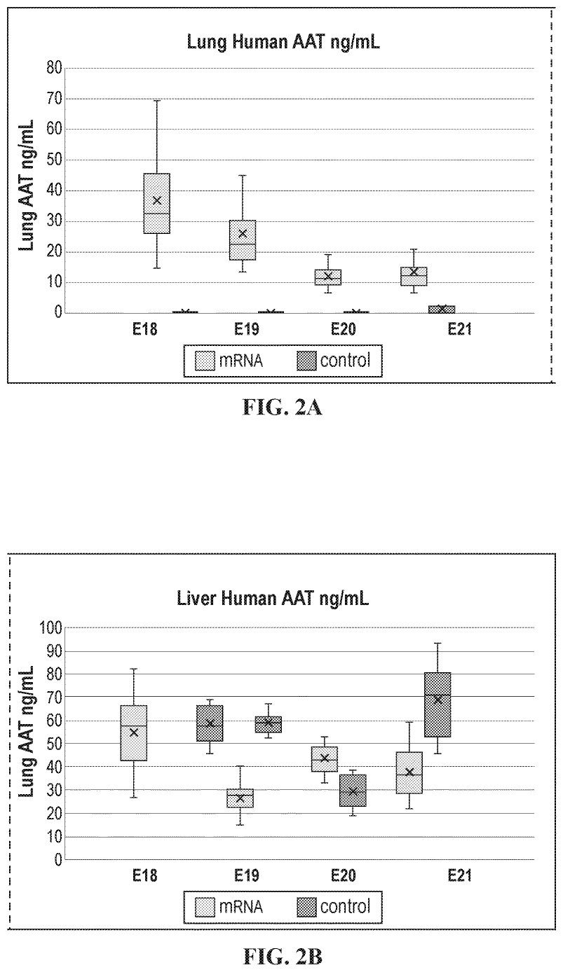

When controlled by mRNA-free injections, there was evidence of human AAT production in the fetal lungs at all four daily time points (p=0.005 to <0.001; FIG. 2A). The levels of human AAT in the lungs were highest at E18-median 32.6 (26.2, 45.6) and tended to decrease thereafter, stabilizing one day before and at term (FIG. 2A, Table 1). The results of pairwise comparisons when accounting for nesting within dams were as follows: E18 p<0.001, E19 p<0.001, E20 p<0.001, and E21 p<0.005, all four of which demonstrated higher levels in the mRNA group.

Controlled by mRNA-free injections, human AAT was detected in the fetal liver only at E20 (p<0.001), with levels significantly higher in the control group at E19 and E21 (p<0.001 and p<0.003 respectively), suggesting possible interspecies homology manifesting at this anatomical site (FIG. 2A, Table 1).

TABLE 1
AAT Protein Levels by ELISA in the Liver and Lungs
on each gestational day (E) after injection

| Variable | Control | | mRNA | | P value |
|---|---|---|---|---|---|
| E18 | n = 9 | | n = 21 | | — |
| Liver (ng/mL) | 57.5 | (53.1, 66.2) | 57.7 | (43.3, 65.7) | 0.991 |
| Lung (ng/mL) | 0 | (0, 0) | 32.6 | (26.2, 45.6) | <0.001* |
| E19 | n = 7 | | n = 22 | | |
| Liver (ng/mL) | 58 | (52.6, 60) | 27.6 | (22.7, 29.9) | <0.001* |
| Lung (ng/mL) | 0 | (0, 0) | 23.6 | (20, 29.2) | <0.001* |
| E20 | n = 5 | | n = 21 | | |
| Liver (ng/mL) | 29.2 | (27.4, 34.2) | 43 | (38.5, 48.1) | <0.001* |
| Lung (ng/mL) | 0 | (0, 0) | 11.2 | (9.5, 14) | <0.001* |
| E21 | n = 6 | | n = 19 | | |
| Liver (ng/mL) | 71 | (55.3, 76.5) | 36.5 | (28.6, 46.3) | 0.003* |
| Lung (ng/mL) | 0 | (0, 0) | 12.1 | (9, 13.5) | 0.005* |

P values were calculated using median regression with a robust standard error to account for nesting of fetuses within mothers. Data presented as median and IQR.
*Statistically significant (p < 0.05).

No difference was detected for hAAT between mRNA and control fetuses for liver samples at E18.

For both lung and liver samples, there was no difference in statistical significance when accounting or not for nesting of fetuses within the same dam.

Standard liver panels at term were comparable between the groups with no statistical difference for all panel elements except for albumin and gamma-glutamyl transferase. The control group had higher albumin [1.9 (1.4, 2.2) g/dL] and total cholesterol [27 (21,32) mg/dL] levels than the mRNA group [1 (1, 1.1) g/dL and 20 (20,33) mg/dL respectively], both p<0.001.

Pulmonary pathology in AATD stems from uninhibited serine protease activity, most notably neutrophil elastase, leading to parenchymal damage and eventually emphysema. Liver pathology results from the accumulation of misfolded AAT protein within the endoplasmic reticulum of hepatocytes causing local cell death and eventually cirrhosis. Treatment for AATD lung disease includes infusions of pooled human plasma containing AAT, which may reduce inflammation and slow disease progression but are expensive and typically have to be administered weekly. Lung transplantation is considered in severe cases to improve quality of life. The only current treatment for AATD-related liver cirrhosis is transplantation, though experimental work suggests a potential benefit from AAT augmentation therapy by downregulating wildtype SerpinA1 expression in a dose-dependent manner.

A few studies have demonstrated the therapeutic potential of encapsulated mRNA delivery and subsequent translation of the desired protein product in murine models of metabolic disease such as acute intermittent porphyria and arginase deficiency. The benefit of mRNA therapy is unique in that it involves modifications of the native cell translational machinery without nuclear manipulations or risk of insertional mutagenesis. SerpinA1-encoding mRNA delivered ex-vivo to fibroblast-derived hepatocytes from patients with AAT has been shown to lead to SerpinA1 protein expression in the cell culture media. That same encapsulated mRNA delivered to wild type mice intravenously have led to the expression of the exogenous protein in the lungs and liver. Delivery of mRNA encoding for hAAT and protein production in cultured human hepatocytes from both healthy and AATD patients has also been reported. That same mRNA delivered to wild type and NSG-PiZ transgenic mouse models of AATD have resulted in hepatic uptake, translation and functionality of the hAAT.

Delivery of AAT mRNA to the fetus had yet to be described until the present work. A study from 1981 showed that the AATD phenotype was detectable in the fetal plasma by mid-gestation, suggesting a potential utility for an eventual form of fetal therapy. Indeed, neonates may present with prolonged cholestatic jaundice and transaminitis. Most of these cases resolve on their own, however a small subset of infants may develop cirrhosis, portal hypertension, and eventually liver failure requiring transplantation. The present examples support the notion that the minimally invasive transamniotic route may be a viable alternative for fetal delivery of hAAT mRNA, with sustained protein translation by the host.

Given that most of the AATD manifestations in the newborn are in the liver, a limitation of this data was the evidence of interspecies homology in that organ, which prevented researchers from discerning an eventual presence of the hAAT protein therein. Certainly, nearly 70% homology between rat and human AAT has long been identified. The above mentioned previous work on systemic AAT mRNA delivery in adult mice, however, would suggest that delivery to the fetal liver seems feasible. During the first ten weeks of gestation the liver and yolk sac both produce AAT, after which time the contribution from the yolk sac quickly weans until being no longer detectable. However, it is not clear when in gestation or childhood AAT begins to appear in the lungs. Given the major changes that lungs undergo after birth, it is not unreasonable to speculate that AAT may be present in the liver in fetal life but not in the lungs until after birth, which could possibly explain a homology discrepancy between the liver and lungs in these examples.

It was beyond the scope of the present examples to delve into the functionality of the AAT product. The comparable results in the hepatic panel between mRNA and control groups would suggest safety of TRANAT for AAT delivery.

Considering the many limitations of current AAT augmentation therapy and typical ultimate need for transplantation(s), new strategies for AAT replacement are sorely needed. The present examples demonstrate that encapsulated exogenous mRNA encoding for hAAT can be incorporated and translated by fetal lung cells following simple intra-amniotic injection in a healthy rat model. Fetal hepatic incorporation and translation remains to be determined in a model with minimal to no human homology. Transamniotic mRNA delivery is likely a novel strategy for the perinatal management of AATD.

The results described herein above were carried out using the following methods and materials.

mRNA Formulation and Encapsulation

A specific human AAT (hAAT) mRNA sequence was custom-ordered and obtained commercially (NCBI: M11465.1; Ribo Pro, The Netherlands). Said sequence was further modified to decrease immunogenicity by adding Cap 1 to 5′-end, as well as 150 nt PolyA-tail to 3′-end and additional proprietary sequence modifications to enhance translation efficiency and mRNA stability (RiboPro). In order to further enhance mRNA stability and facilitate in vivo delivery, nanoparticles consisting of a self-assembling composite lipid- and synthetic cationic polymer-based lipopolyplex were used for encapsulation of the hAAT mRNA using a mRNA transfection kit (TransIT; Mirus Bio, Madison) made of two components, a reagent proper and an mRNA boost reagent, in accordance with the manufacturer's instructions. In brief, 1 μg of mRNA (1 μg/μL stock) was suspended in 45 μL of phosphate-buffered saline (PBS). Subsequently, 2 μL of reagent proper and 2 μL of mRNA boost reagent were added to the suspension followed by incubation for 5 to 10 minutes at room temperature to allow for encapsulation.

Encapsulation efficiency of the hAAT mRNA into the lipopolyplex was measured using the Quant-iT RiboGreen assay (Thermo Fisher Scientific, Waltham, MA) as previously described. Briefly, known amounts of encapsulated mRNA into lipopolyplex (1 μg/50 μL stock) were mixed with fluorescent RiboGreen reagent (Thermo Fisher Scientific). The amount of free-floating mRNA in that solution was then measured on a microplate reader (BMG Labtech, Cary, NC) by fluorescent detection at 490 nm and 520 nm excitation and emission, respectively. Encapsulation efficiency was determined by subtracting the amount of free mRNA in the lipopolyplex solution (lipopolyplex mRNA) from the total mRNA originally used (naked mRNA) and expressed as a percentage. The same procedure was performed in parallel using naked mRNA in PBS (1 μg/50 μL stock) instead of encapsulated mRNA, as positive controls.

### Intra-Amniotic Injections

Twelve Sprague Dawley dams (Charles River Laboratories, Wilmington, MA) underwent surgical intra-amniotic injections under direct visualization on gestational day 17 (E17, term=E21) as previously described. In brief, animals were anesthetized by 2-4% inhaled isoflurane (Patterson Veterinary, Greeley, CO) in 100% oxygen. A midline laparotomy was performed to expose the bicornuate uterus. All viable fetuses (n=139) received volume-matched (50 μL) intra-amniotic injections of either a suspension of a hAAT mRNA encapsulated as described above (mRNA group; n=99), or of a suspension of the lipopolyplex free of mRNA (control group; n=40). All injections were performed using a 33G non-coring needle on a 100 μL syringe (both from Hamilton Company, Reno, NV) under direct visualization taking care to avoid injuring the fetus, umbilical cord, or placenta. The laparotomy was then closed in two layers and powdered metronidazole (Unichem Pharmaceuticals, Hasbrouck Heights, NJ) was applied to the wound. Sustained-release buprenorphine (Fidelis Pharmaceuticals, North Brunswick, NJ) was administered for post-operative analgesia.

### Specimen Procurement

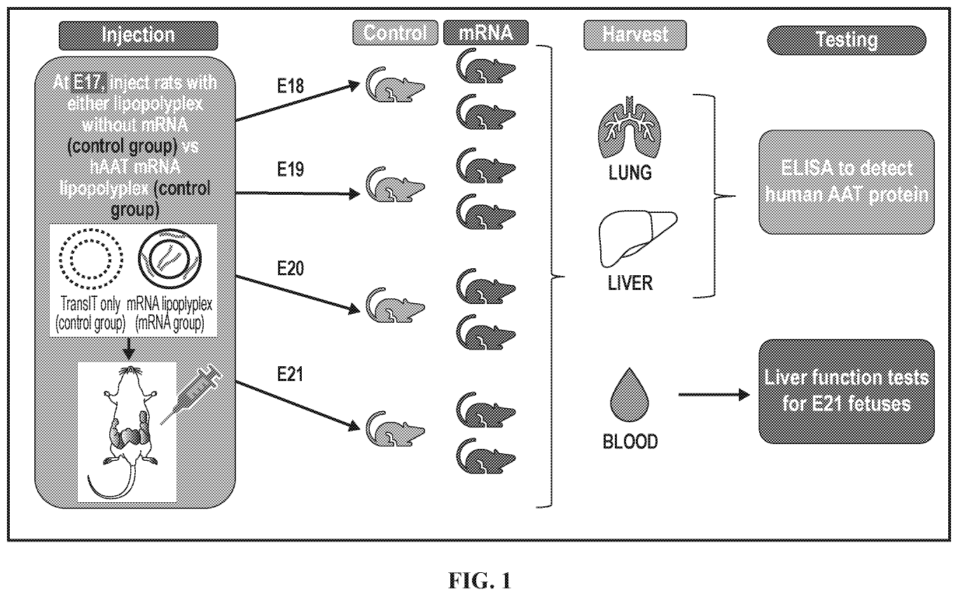

Dams were euthanized by CO₂ chamber at daily time points from E18 to term at E21 distributed as two dams per gestational age for the mRNA group and one dam per gestational age for the control group (FIG. 1). The laparotomy was reopened, and the uterus was eviscerated. Each fetus was removed en caul followed by procurement of their lungs and livers. A control dam from E21 delivered just prior to the scheduled euthanasia, so its fetuses were sacrificed by decapitation immediately after delivery, followed by lung and liver samples procurement. All lung and liver specimens were frozen by rapid dry ice-ethanol baths and stored at −80° C. until further processing. Peripheral blood samples were procured from fetuses at the E21 time-point only underwent standard liver panel analysis (details below).

hAAT ELISA

Lung and liver samples were standardized by weight, washed in PBS, and homogenized in 0.1M PBS pH 7.4 containing 1% Triton X-100 with stainless steel beads (Next Advance, Averill Park, NY) in an automated tissue homogenizer (Next Advance) for 8 minutes followed by 1 hour on an orbital shaker at 4° C. to ensure complete extraction. Specimen homogenates were then centrifuged at 14,000×g for 20 minutes, and the supernatant was then used at a 1:5 dilution for liver samples and mostly 1:5 dilution or 1:10 for smaller lung samples for hAAT detection using a commercially available hAAT ELISA kit (ab108799, Abcam, Waltham, MA) according to the manufacturer's instructions. The final hAAT concentration was calculated using a standard curve after reading the absorbance at 450 nm wavelength on a microplate reader (BMG Labtech, Cary, NC) expressed in ng/mL. All samples and standards were run in duplicates.

### Liver Enzymes

Blood samples from E21 fetuses (control n=4, mRNA n=13) were immediately transferred into lithium heparin-coated green top test tubes. Two samples from the control group had coagulated blood upon attempted retrieval, therefore samples from only four fetuses could be analyzed. Two samples from the mRNA group samples were excluded for the same reason. Standard liver panel analyses included alkaline phosphate, alanine aminotransferase, gamma-glutamyl transferase, bile acids, total bilirubin, albumin, blood urea nitrogen, total cholesterol measurements produced using the Mammalian Liver Profile Rotor (Zoetis, Abaxis, Inc; Union City, CA) according to the manufacturer's instructions. Each rotor was loaded individually into the VetScanVS2 (Zoetis, Abaxis, Inc), a portable chemistry analyzer based on rotor technology.

### Statistical Analyses

Data were compared by non-parametric Wilcoxon rank sum test and median regression to account for nesting within dams in Stata 16.1 (StataCorp, College Station, TX). Statistical significance was set at p<0.05.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for treating alpha-1 antitrypsin deficiency (AATD) in a developing fetus, the method comprising: administering to the amniotic fluid surrounding a developing fetus an mRNA molecule encoding an alpha-1 antitrypsin (AAT) polypeptide, thereby treating AATD in the developing fetus.

2. The method of claim 1, wherein the mRNA is translated in the lungs and/or liver of the fetus to produce the AATD polypeptide in lung cells of the fetus.

3. The method of claim 1, wherein the method comprises reducing the incidence of chronic obstructive pulmonary disease in the fetus or the child developed from the fetus.

4. A method for treating a disease or disorder associated with an AAT deficiency in a developing fetus, the method comprising: administering to the amniotic fluid surrounding a developing fetus an mRNA molecule encoding an AAT polypeptide, thereby treating the disease or disorder associated with AAT deficiency in the developing fetus.

5. The method of claim 4, wherein the disease or disorder is prematurity or congenital AAT deficiency.

6. The method of claim 4, wherein the mRNA is translated in the liver of the fetus to produce the AAT polypeptide in liver cells of the fetus.

7. The method of claim 4, wherein the mRNA is translated in lung cells of the fetus to produce the AAT polypeptide in airway and/or pulmonary cells of the fetus.

8. The method of claim 4, wherein the method is associated with an increase in levels of AAT in the amniotic fluid.

9. The method of claim 1, wherein the mRNA is administered using a lipid nanoparticle or a lipopolyplex.

10. The method of claim 1, wherein the mRNA is codon-optimized.

11. The method of claim 1, wherein the mRNA is administered prior to 10 weeks of pregnancy.

12. The method of claim 1, wherein the mRNA is administered prior to 5 weeks of pregnancy.

13. The method of claim 1, wherein the mRNA is administered after 10 weeks of pregnancy.

14. The method of claim 1, wherein the administering is associated with the presence of and/or an increase in levels of the mRNA in a tissue of the fetus.

15. The method of claim 14, wherein the tissue comprises liver, stomach, intestines, pancreas, spleen, thymus, lymph nodes, brain, meninges, heart, blood vessels, lungs, airways, kidneys, ureters, urethra, ovaries, testicles, genitalia, skin, skin annexes, muscle, bone, cartilage, bone marrow, eyes, ears, mouth, nose, pharynx, larynx, vitreous body, cerebrospinal fluid, and peripheral blood.

16. The method of claim 15, wherein the tissue comprises liver, stomach, intestines, lungs, and/or blood.

17. The method of claim 1, wherein the administration is associated with the presence of and/or an increase in levels of the mRNA in amniotic fluid, amnion, chorion, umbilical cord, and/or placenta associated with the fetus.

18. A kit suitable for use in carrying out the method of claim 1.

19. A method for delivering one or more mRNA molecules to the bloodstream of a developing fetus, the method comprising: administering to the amniotic fluid surrounding the developing fetus an mRNA molecule encoding a polypeptide that has reduced expression and/or activity in the developing fetus relative to a healthy developing fetus.

20. The method of claim 19, wherein the mRNA is translated in an organ and/or tissue of the developing fetus, thereby treating the developing fetus for a disease or disorder associated with the polypeptide.


SEQUENCE LISTING (2 sequences)

SEQ ID NO: 1 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - DNA, 3243 bp, Homo sapiens
  source 1..3243: mol_type = genomic DNA
agagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca